(19) United States
(12) Patent Application Publication
(10) Pub. No.: US 2025/0302310 A1
(43) Pub. Date: Oct. 2, 2025

(54) POSITIONING ASSISTANCE METHOD AND MEDICAL IMAGING SYSTEM

(71) Applicant: GE Precision Healthcare LLC, Waukesha, WI (US)

(72) Inventors: Minghui Ye, Beijing (CN); Yingying Wang, Beijing (CN); Yannan Huang, Beijing (CN); Mingtao Hu, Beijing (CN)

(21) Appl. No.: 19/098,457

(22) Filed: Apr. 2, 2025

(30) Foreign Application Priority Data
Apr. 2, 2024 (CN) 202410392895.1

(51) Int. Cl. A61B 5/00; A61B 5/11

(52) U.S. Cl. CPC A61B 5/0077; A61B 5/1114

(57) ABSTRACT

Provided in embodiments of the present application are a positioning assistance method and a medical imaging system. The positioning assistance method includes: obtaining image data captured via an image capture apparatus and including a subject, the image data including optical image data and depth image data; determining, according to the image data, a direction indication line representing at least one anatomical region of interest; and calculating a body region inclination angle according to the direction indication line, the body region inclination angle including at least one of a first included angle and a second included angle, the first included angle representing an included angle between an anatomical region of interest and a plane on which hardware of a medical imaging system is located, and the second included angle representing an included angle between two anatomical regions of interest.

### CROSS-REFERENCE TO RELATED APPLICATIONS

The present application claims priority to Chinese Application No. 202410392895.1, filed on Apr. 2, 2024, the entire contents of which is hereby incorporated by reference.

### TECHNICAL FIELD

Embodiments of the present application relate to the technical field of medical imaging, and relate in particular to a positioning assistance method and a medical imaging system.

### BACKGROUND

In a medical imaging system, emitted X-rays from an X-ray source are directed at a subject and are received by a detector after penetrating the subject. The detector is divided into a matrix of discrete elements (such as pixels). The elements of the detector are read to produce an output signal based on the amount or intensity of radiation impacting each pixel area. The signal is processed to produce a medical image of the subject, and the medical image may be displayed in a display apparatus of the medical imaging system.

### SUMMARY

Provided in embodiments of the present application are a positioning assistance method and a medical imaging system.

According to an aspect of the embodiments of the present application, there is provided a positioning assistance method, comprising:
obtaining image data captured via an image capture apparatus and comprising a subject, the image data comprising optical image data and depth image data;
determining, according to the image data, a direction indication line representing at least one anatomical region of interest; and
calculating a body region inclination angle according to the direction indication line, the body region inclination angle comprising at least one of a first included angle and a second included angle, the first included angle representing an included angle between an anatomical region of interest and a plane on which hardware of a medical imaging system is located, and the second included angle representing an included angle between two anatomical regions of interest.

According to an aspect of the embodiments of the present application, there is provided a medical imaging system, comprising:
an image capture apparatus, capturing a subject to obtain image data comprising the subject, the image data comprising optical image data and depth image data; and
a control apparatus, connected to the image capture apparatus and configured to perform the positioning assistance method in the foregoing aspect.

According to an aspect of the embodiments of the present application, there is provided a non-transitory computer-readable storage medium, comprising at least a computer program, the computer program, when executed by a processor, performing the positioning assistance method in the foregoing aspect.

With reference to the following description and drawings, specific implementations of the embodiments of the present application are disclosed in detail, and the way in which the principles of the embodiments of the present application can be employed are illustrated. It should be understood that the embodiments of the present application are not limited in scope thereby. Within the scope of the spirit and clauses of the appended claims, the embodiments of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the embodiments of the present application and explain the principles of the present application together with textual description. Evidently, the drawings in the following description are merely some embodiments of the present application, and those of ordinary skill in the art may obtain other embodiments based on the drawings without involving inventive effort. In the drawings:

FIG. 1 is a schematic diagram of a medical imaging system according to an embodiment of the present application;

FIG. 2 is a schematic diagram of special positioning according to an embodiment of the present application;

FIG. 3 is a schematic diagram of a positioning assistance method according to an embodiment of the present application;

FIG. 4 is a schematic diagram of a method for calculating a second included angle according to an embodiment of the present application;

FIG. 5 is a schematic diagram of two direction indication lines in a world coordinate system according to an embodiment of the present application;

FIG. 6 is a schematic diagram of a method for calculating a first included angle according to an embodiment of the present application;

FIG. 7 shows an area matching a preset mask according to an embodiment of the present application;

FIG. 8 is a top view of two direction indication lines according to an embodiment of the present application;

FIG. 9 is a schematic diagram of a medical imaging method according to an embodiment of the present application;

FIG. 10 is a schematic diagram of a positioning assistance apparatus according to an embodiment of the present application; and

FIG. 11 is a schematic diagram of a medical imaging system according to an embodiment of the present application.

### DETAILED DESCRIPTION

The foregoing and other features of the embodiments of the present application will become apparent from the following description with reference to the drawings. In the description and drawings, specific embodiments of the present application are disclosed in detail, and some of the embodiments in which the principles of the embodiments of the present application may be employed are indicated. It should be understood that the present application is not limited to the described embodiments. On the contrary, the embodiments of the present application include all modifications, variations, and equivalents which fall within the scope of the appended claims.

In the embodiments of the present application, the terms “first”, “second”, etc., are used to distinguish between different elements in terms of appellation, but do not represent a spatial arrangement, a temporal order, or the like of these elements, and these elements should not be limited by these terms. The term “and/or” includes any one of and all combinations of one or more associated listed terms. The terms “include”, “comprise”, “have”, etc., refer to the presence of described features, elements, components, or assemblies, but do not exclude the presence or addition of one or more other features, elements, components, or assemblies. The terms “connect”, “link”, “couple”, etc., used in the embodiments of the present application are not limited to physical or mechanical connections, but may include electrical connections, whether direct or indirect.

In the embodiments of the present application, the singular forms “a/an” and “the”, etc., include plural forms, and should be broadly construed as “a type of” or “a class of” rather than being limited to the meaning of “one”. In addition, the term “the” should be construed as including both the singular and plural forms, unless otherwise explicitly specified in the context. In addition, the term “according to” should be construed as “at least in part according to . . . ”, and the term “based on” should be construed as “based at least in part on . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

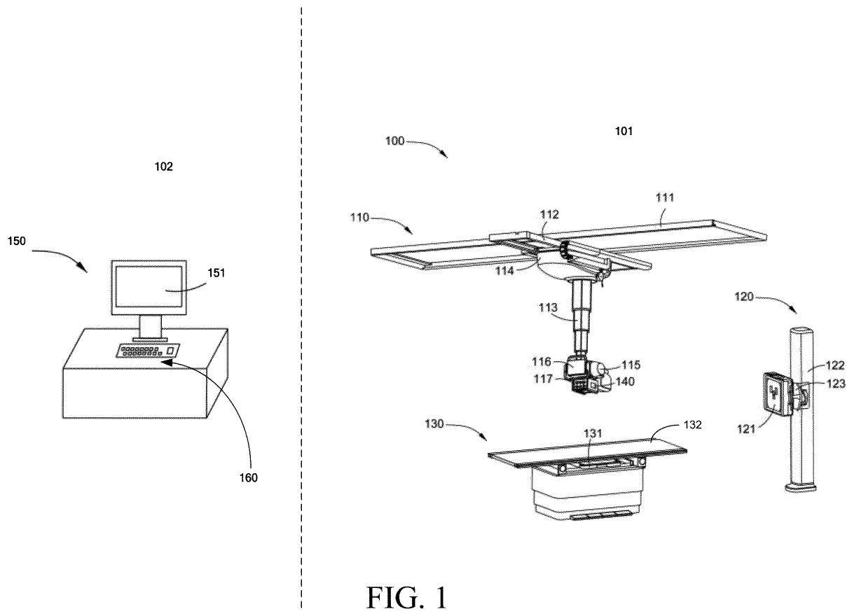

FIG. 1 is a medical imaging system 100 according to an embodiment of the present application. As shown in FIG. 1, the medical imaging system 100 includes a suspension apparatus 110, a wall stand apparatus 120, and an examination table apparatus 130 arranged in a scanning room 101, and a control apparatus 150 arranged in a control room 102. The suspension apparatus 110 includes a longitudinal guide rail 111, a transverse guide rail 112, a telescopic cylinder 113, a sliding member 114, and a tube assembly 115.

Although some embodiments of the present application are described based on a suspended X-ray imaging system, the embodiments of the present application are not limited thereto.

For ease of description, in the present application, an x-axis, a y-axis, and a z-axis are defined as the x-axis and the y-axis being located on a horizontal plane and being perpendicular to one another, and the z-axis being perpendicular to the horizontal plane. Specifically, the direction in which the longitudinal guide rail 111 is located is defined as the x-axis, the direction in which the transverse guide rail 112 is located is defined as the y-axis direction, and the direction of extension of the telescopic cylinder 113 is defined as the z-axis direction, and the z-axis direction is the vertical direction.

The longitudinal guide rail 111 and the transverse guide rail 112 are perpendicularly arranged, wherein the longitudinal guide rail 111 is mounted on a ceiling, and the transverse guide rail 112 is mounted on the longitudinal guide rail 111. The telescopic cylinder 113 is used to carry the tube assembly 115.

The sliding member 114 is arranged between the transverse guide rail 112 and the telescopic cylinder 113. The sliding member 114 may include components such as a rotary shaft, a motor, and a reel. The motor can drive the reel to rotate around the rotary shaft, which in turn drives the telescopic cylinder 113 to move along the z-axis and/or slide relative to the transverse guide rail. The sliding member 114 can slide relative to the transverse guide rail 112, that is, the sliding member 114 can drive the telescopic cylinder 113 and/or the tube assembly 115 to move in the y-axis direction. Furthermore, the transverse guide rail 112 can slide relative to the longitudinal guide rail 111, which in turn drives the telescopic cylinder 113 and/or the tube assembly 115 to move in the x-axis direction.

The telescopic cylinder 113 includes a plurality of columns having different inner diameters, and the plurality of columns may be sleeved sequentially from bottom to top in columns located thereon to thereby achieve telescoping. The telescopic cylinder 113 can be telescopic (or movable) in the vertical direction, that is, the telescopic cylinder 113 can drive the tube assembly to move in the z-axis direction. The lower end of the telescopic cylinder 113 is further provided with a rotating part, and the rotating part may drive the tube assembly 115 to rotate.

The tube assembly 115 includes an X-ray tube, and the X-ray tube may produce X-rays and project the X-rays to a patient's intended region of interest (ROI). Specifically, the X-ray tube may be positioned adjacent to a beam limiter, and the beam limiter is used to align the X-rays with the patient's intended region of interest. At least part of the X-rays may be attenuated by means of the patient and may be incident on a detector 121/131. In addition, not shown in the drawings, the X-ray imaging system may further include a positionally flexible hand-held detector for imaging some joints or infants.

The suspension apparatus 110 further includes a beam limiter 117, which is usually mounted below the X-ray tube, and the X-rays emitted by the X-ray tube irradiate on the body of a subject through an opening of the beam limiter 117. The size of the opening of the beam limiter 117 determines an irradiation range of the X-rays, namely, the size of an area of an exposure field of view (FOV). The positions of the X-ray tube and beam limiter 117 in the transverse direction determine the position of the exposure FOV on the body of the subject. It is well known that X-rays are harmful to the human body, so it is necessary to control the X-rays so that the X-rays only irradiate the region of the subject that needs to be examined, namely, the region of interest (ROI).

The suspension apparatus 110 further includes a tube control apparatus (console) 116. The tube control apparatus 116 is mounted on the tube assembly. The tube control apparatus 116 includes user interfaces such as a display screen and a control button for performing preparation work before image capture, such as patient selection, protocol selection, positioning, etc.

The movement of the suspension apparatus 110 includes the movement of the tube assembly along the x-axis, y-axis, and z-axis, as well as the rotation of the tube assembly on a horizontal plane (the axis of rotation is parallel to or coincides with the z-axis) and on a vertical plane (the axis of rotation is parallel to the y-axis). In the described movement, a motor is usually used to drive a rotary shaft which in turn drives a corresponding component to rotate, so as to achieve a corresponding movement or rotation, and a corresponding control component is generally mounted in the sliding member 114. An X-ray imaging unit further includes a motion control unit (not shown in the figure), and the motion control unit can control the described movement of the suspension apparatus 110. Further, the motion control unit can receive a control signal to control a corresponding component to move accordingly.

The wall stand apparatus 120 includes a first detector assembly 121, a wall stand (for example, a chest radiography stand) 122, and a connecting portion 123. The connecting portion 123 includes a support arm that is vertically connected in the height direction of the wall stand 122 and a rotating bracket that is mounted on the support arm, and the first detector assembly 121 is mounted on the rotating bracket. The wall stand apparatus 120 further includes a detector driving apparatus that is arranged between the rotating bracket and the first detector assembly 121. Under the driving of the detector driving apparatus, the first detector assembly 121 moves in a direction that is parallel to the height direction of the wall stand 122 on a plane that is supported by the rotating bracket, and the first detector assembly 121 may be further rotated relative to the support arm to form a specific angle with the wall stand. The first detector assembly 121 has a plate-like structure the orientation of which can be changed, so that the incident surface of the X-rays becomes vertical or horizontal depending on the incident direction of the X-rays.

The examination table apparatus 130 includes a bedplate 132 and a second detector assembly 131. The selection or use of the first detector assembly 121 and the second detector assembly 131 may be determined based on an image capture region of a patient and/or an image capture protocol, or may be determined based on the position of the subject that is obtained by the capturing of a camera, so as to perform image capture and examination at a supine, prone, or standing position. FIG. 1 is merely a schematic diagram of a wall stand and an examination table. It should be understood by those skilled in the art that a wall stand and/or an examination table in any form or arrangement may be selected or only the wall stand may be mounted. The wall stand and/or the examination table do/does not limit the entire solution of the present application.

In some embodiments, the control apparatus 150 may include a source controller and a detector controller. The source controller is configured to command the X-ray source to emit X-rays for image exposure. The detector controller is configured to select a suitable detector among a plurality of detectors, and to coordinate the control of various detector functions, such as automatically selecting a corresponding detector based on the position or posture of the subject. Alternatively, the detector controller may perform various signal processing and filtering functions, specifically, for initial adjustment of a dynamic range, interleaving of digital image data, and the like. In some embodiments, the control apparatus may provide power and timing signals for controlling the operation of the X-ray source and the detector.

In some embodiments, the control apparatus may alternatively be configured to use a digitized signal to reconstruct one or more required images and/or determine useful diagnostic information corresponding to a patient, wherein the control apparatus may include one or more dedicated processors, graphics processing units, digital signal processors, microcomputers, microcontrollers, application-specific integrated circuits (ASICs), field-programmable gate arrays (FPGAs), or other suitable processing apparatuses.

Certainly, the medical imaging system may further include other numbers, configurations or forms of control apparatuses, for example, the control apparatus may be local (for example, co-located with one or more medical imaging systems 100, such as within the same facility and/or the same local network). In other implementations, the control apparatus may be remote, and thus only accessible through a remote connection (for example, via the Internet or other available remote access technologies). In a specific implementation, the control apparatus may alternatively be configured in a cloud-like manner, and may be accessed and/or used in a manner that is substantially similar to a manner of accessing and using other cloud-based systems.

The system 100 further includes a storage apparatus (not shown in the figure). A processor may store the digitized signal in a memory. For example, the memory may include a hard disk drive, a floppy disk drive, a CD-read/write drive, a digital versatile disc (DVD) drive, a flash drive, and/or a solid-state memory. The memory may alternatively be integrated together with the processor to effectively use the footprint and/or meet expected imaging requirements.

The system 100 further includes an input apparatus 160. The input apparatus 160 may include a specific form of operator interface, such as a keyboard, a mouse, a voice-activated control apparatus, a touchscreen (which may also be used as a display apparatus described later), a trackball, or any other suitable input device. An operator may input an operation signal/control signal to the control apparatus by using the input device.

The system 100 further includes a display apparatus 151 (such as a touchscreen or a display screen). The display apparatus 151 may be configured to display an operation interface such as a list of subjects, the positioning or exposure settings of the subjects, and images of the subjects.

In some embodiments, the medical imaging system may further include an image capture apparatus 140. The subject may be captured by using the image capture apparatus to obtain a captured image including the subject, for example, a still image or a series of image frames in a dynamic real-time video stream, to perform positioning assistance, exposure setting, and the like. The image capture apparatus may be mounted on the suspension apparatus, for example, be mounted on a side edge of the beam limiter 117, and the embodiments of the present application are not limited thereto.

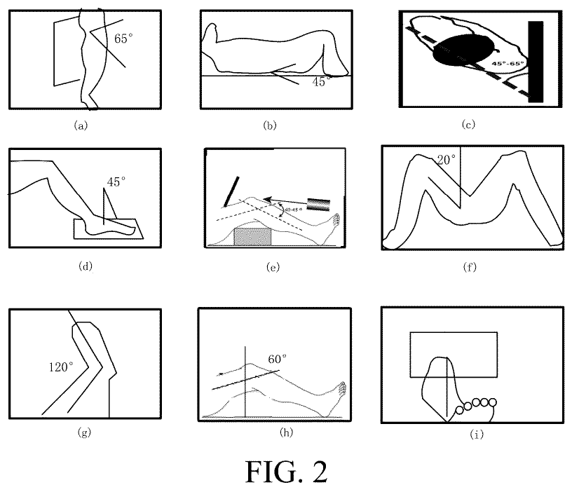

When an existing medical imaging system is in use, the subject needs to be positioned correctly. In particular, to more effectively and accurately diagnose some types of trauma, some special positions (positioning with an angle) are required. For example, FIG. 2(a) shows positioning of a hip joint inclined 65°, that is, the hip joint is not positioned horizontally against a wall stand apparatus 120, but inclined 65° relative to a detector 121 (a wall stand plane) on the wall stand. FIG. 2(b) shows positioning of a lumbar vertebra inclined 45°, that is, the lumbar vertebra is not horizontally located on a bedplate, but inclined 45° relative to the bedplate (a detector 131 under the bedplate). FIG. 2(c) shows a Y side position of a shoulder joint, that is, a scapula line is inclined 45° to 65° relative to the detector 121 on the wall stand. FIG. 2(d) shows positioning of a foot inclined 45°, that is, the sole is inclined 45° relative to the detector in a flat panel free mode of the detector. FIG. 2(e) shows positioning of a thigh and a leg at an included angle of 40° to 45° in a side view. FIG. 2(f) shows positioning of a thigh inclined 20° relative to the normal of the bedplate (the detector 131 under the bedplate) in a bottom view. FIG. 2(g) shows positioning of an ankle and a foot at an angle of 120°. FIG. 2(h) shows positioning of a thigh inclined 60° relative to the bedplate (the detector 131 under the bedplate) in a side view. FIG. 2(i) shows a line of a patella apex and a tibiofibular bone perpendicular to a handheld detector plane.

Currently, visual measurement methods are mostly used in clinical practice, and positioning assistance is prompted depending on the experience of an operator. Alternatively, positioning assistance such as a set square may be used. However, such a positioning method is not accurate enough, takes a long time, and lacks accurate quantitative information, and it is often difficult to accurately obtain a clinically required angle through only one exposure.

To address at least one of the above problems, the embodiments of the present application provide a positioning assistance method and a medical imaging system. The embodiments of the present application are described below in detail.

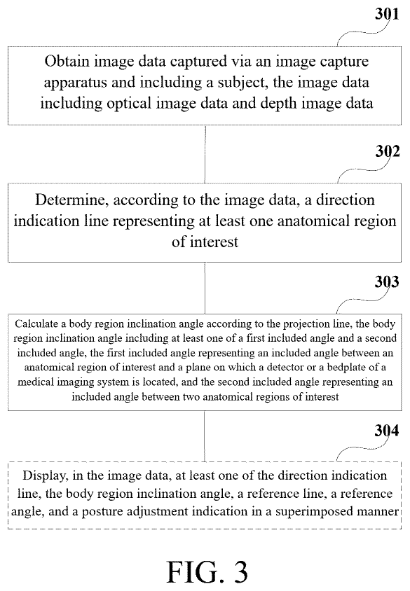

Provided in the embodiments of the present application is a positioning assistance method. FIG. 3 is a schematic diagram of a positioning assistance method according to an embodiment of the present application. As shown in FIG. 3, the method includes:
301: obtaining image data captured via an image capture apparatus and including a subject, the image data including optical image data and depth image data;
302: determining, according to the image data, a direction indication line representing at least one anatomical region of interest; and
303: calculating a body region inclination angle according to the direction indication line, the body region inclination angle including at least one of a first included angle and a second included angle, the first included angle representing an included angle between an anatomical region of interest and a plane on which hardware of a medical imaging system is located, and the second included angle representing an included angle between two anatomical regions of interest.

In some embodiments, image data captured by one or more image capture apparatuses may be obtained. For example, the image capture apparatuses may include devices such as a digital camera and an analog camera, or a depth camera, an infrared camera, and an ultraviolet camera, or a 3D camera and a 3D scanner, or a red, green, and blue (RGB) sensor and an RGB depth (RGB-D) sensor. The image data may include optical image data and depth image data. The optical image may be a two-dimensional RGB image, and each pixel value of the depth image reflects a distance between the image capture apparatus and a position corresponding to the subject. The image data may be one frame of a still image captured by the image capture apparatus, or any frame of image in a dynamic real-time video stream. Embodiments of the present application are not limited thereto.

In some embodiments, in scenarios of some special positions (positioning with an angle), at least one of the first included angle and the second included angle may be used to represent a position of the subject. The first included angle represents the included angle between the anatomical region of interest and the plane on which the hardware of the medical imaging system is located. The hardware includes, but is not limited to, a detector, a bedplate, a wall stand, and the like. For example, the first included angle is the included angles shown in FIG. 2(a), FIG. 2(b), FIG. 2(c), FIG. 2(d), FIG. 2(f), FIG. 2(h), and FIG. 2(i), as described above. The second included angle represents an included angle between two anatomical regions of interest, such as the included angles shown in FIG. 2(e) and FIG. 2(g), as described above.

In some embodiments, the type of the included angle that needs to be calculated may be determined according to a scanning protocol (or a region to be exposed) for a special position. For example, the first included angle (and the number thereof) or the second included angle (and the number thereof) may be determined and calculated according to the scanning protocol (or the region to be exposed) for the special position. Alternatively, the first included angle and the second included angle may need to be calculated to assist in positioning.

For example, when the scanning protocol is scanning of a special position of a knee joint, to assist in positioning, two first included angles shown in FIG. 2(f) and FIG. 2(h) need to be calculated. For example, when the scanning protocol is a patella axis—sunrise, to assist in positioning, the second included angle shown in FIG. 2(e) and the first included angle shown in FIG. 2(i) need to be calculated. For example, the scanning protocol is a Y side position of a scapula. To assist in positioning, the first included angle shown in FIG. 2(c) needs to be calculated.

In some embodiments, the number of angle measurement orientations (that is, the movement of the image capture apparatus is preset and controlled) may be determined according to the scanning protocol (or the type of the included angle that needs to be calculated) to obtain appropriate image data for calculating the above included angle.

In some embodiments, the medical imaging system is provided with only one image capture apparatus (which is arranged, for example, on the suspension apparatus). For example, when the scanning protocol is scanning of a special position of a knee joint, two first included angles shown in FIG. 2(f) and FIG. 2(h) need to be calculated. Therefore, two angle measurement orientations (measurement orientation 1 and measurement orientation 2) are required. To be specific, the image capture apparatus (the suspension apparatus) is controlled to move to measurement orientation 1 to obtain image data from a perspective of a bottom view, so as to calculate the first included angle shown in FIG. 2(f). The image capture apparatus (the suspension apparatus) further needs to be controlled to move to measurement orientation 2 to obtain image data from a perspective of a side view, so as to calculate the first included angle shown in FIG. 2(h).

For example, when the scanning protocol is a patella axis—sunrise, the first included angle shown in FIG. 2(e) and the second included angle shown in FIG. 2(i) need to be calculated. Therefore, two angle measurement orientations (measurement orientation 1 and measurement orientation 2) are required. To be specific, the image capture apparatus (the suspension apparatus) is controlled to move to measurement orientation 1 to obtain image data from a perspective of a bottom view, so as to calculate the second included angle shown in FIG. 2(e). The image capture apparatus (the suspension apparatus) further needs to be controlled to move to measurement orientation 2 to obtain image data from a perspective of a side view, so as to calculate the first included angle shown in FIG. 2(i). No further examples are provided herein. The embodiments of the present application set no limitation on the sequence of the measurement orientations.

In some embodiments, the medical imaging system may be provided with a plurality of image capture apparatuses, and the plurality of image capture apparatuses may be respectively arranged in advance at the plurality of measurement orientations described above, without moving the controller. In this way, the image data may be directly obtained, and the above included angles may be calculated for the image data obtained by the plurality of image capture apparatuses. No further examples are provided herein.

In the embodiments of the present application, after the image data is obtained, the above included angles may be calculated according to the image data, as described in detail below.

In some embodiments, at 302, a direction indication line representing at least one anatomical region of interest is determined according to the image data, for example, one direction indication line representing one anatomical region of interest is determined to calculate the first included angle, or two direction indication lines representing two anatomical regions of interest are determined to calculate the second included angle. The direction indication line may reflect an approximate position of the anatomical region of interest, or the direction indication line may represent an approximate extension direction of the anatomical region of interest in a two-dimensional space or a three-dimensional space. The direction indication line may be a vector line representing the anatomical region of interest in the three-dimensional space, or a projection line representing the anatomical region of interest in the two-dimensional space, which will be described in detail later.

In some embodiments, the method may further include: (not shown in the figure) performing matching processing on the optical image data and the depth image data; and determining, according to the matched image data, the direction indication line representing the at least one anatomical region of interest.

In some embodiments, parameters of the image capture apparatus (for example, an intrinsic matrix and an extrinsic matrix of each of a 2D camera and a depth camera) need to be determined. A pixel in the depth image data is converted into a three-dimensional coordinate point A in a world coordinate system by using the parameters of the image capture apparatus. World coordinates of A are projected onto an optical image, coordinates x and y of A on the optical image are obtained, and values of three channels, i.e., RGB of A are extracted and combined with a depth value of A to form four-channel data, to obtain matched image data. The matched image data may alternatively be represented as coordinates (x, y, z), wherein x is a lateral distance, y is a height, and z is a depth value of the depth image data. Embodiments of the present application are not limited thereto.

In some embodiments, the image data may be detected by using a deep learning algorithm to determine an anatomical region of interest in the image data. The anatomical region of interest may be one or more of a plurality of anatomical regions, including, but not limited to, a head, a shoulder, an arm, an elbow, a wrist, a lumbar vertebra, a hip, a knee, a heart, a pelvic cavity, an abdomen, a chest, and an ankle.

For example, an artificial intelligence model may be used to detect an anatomical region of interest from the image data. The artificial intelligence model is implemented based on the deep learning algorithm. For details, reference may be made to the related art. For example, the artificial intelligence model may be a model such as a mask region-based convolutional neural network (mask R-CNN) model, a DensePose model, an OpenPose model, or an HRNet model. The above pre-acquired image data of a plurality of volunteers may be used as an input parameter set, and pre-calibrated key point information corresponding to the image data is used as an output parameter set. The input parameter set and the output parameter set are used to train the artificial intelligence model, and the trained artificial intelligence model is used to detect an anatomical region of interest.

In some embodiments, the type of the model used to calculate the first included angle and the method for obtaining the direction indication line are different from the type of the model used to calculate the second included angle and the method for obtaining the direction indication line. For example, when the first included angle is calculated, the mask R-CNN model and the method for obtaining the direction indication line corresponding to the mask R-CNN model may be used. When the second included angle is calculated, the OpenPose model and the method for obtaining the direction indication line corresponding to the OpenPose model may be used. Only an example is used herein for description, and the embodiments of the present application are not limited thereto.

In other words, the type and number of models used are related to the scanning protocol, and at least two different scanning protocols correspond to different types or numbers of models. Therefore, the type of the model used and the method for obtaining the direction indication line may be preset according to the scanning protocol (or the type of the included angle that needs to be calculated) for the special position.

In some embodiments, when the anatomical region of interest is completely exposed to the image capture apparatus, it is more suitable to use the OpenPose model and the method for obtaining the direction indication line corresponding to the OpenPose model. When the anatomical region of interest is partially occluded, it is more suitable to use the mask R-CNN model and the method for obtaining the direction indication line corresponding to the mask R-CNN model. Only an example is used herein for description, and the embodiments of the present application are not limited thereto.

For example, when the scanning protocol is a special position of an ankle, the second included angle shown in FIG. 2(g) needs to be calculated, and use of the OpenPose model and the method for obtaining the direction indication line corresponding to the OpenPose model need to be preset.

For example, the scanning protocol is a Y side position of a scapula. The first included angle shown in FIG. 2(c) needs to be calculated, and use of the mask R-CNN model and the method for obtaining the direction indication line corresponding to the mask R-CNN model need to be preset.

For example, when the scanning protocol is a patella axis—sunrise, the second included angle shown in FIG. 2(e) and the first included angle shown in FIG. 2(i) need to be calculated. The first included angle shown in FIG. 2(i) is calculated by presetting use of the mask R-CNN model and the method for obtaining the direction indication line corresponding to the mask R-CNN model (for the image data in the above bottom view). The second included angle shown in FIG. 2(e) is calculated by presetting use of the OpenPose model and the method for obtaining the direction indication line corresponding to the OpenPose model (for the image data in the above side view).

How to calculate the first included angle and the second included angle are separately described below.

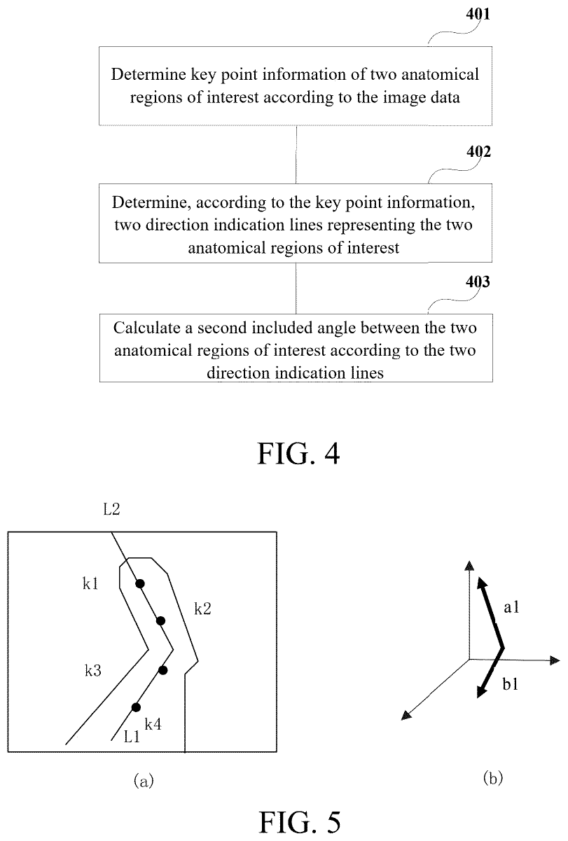

FIG. 4 is a schematic diagram of a method for calculating a second included angle according to an embodiment of the present application. As shown in FIG. 4, the method includes:
401: determining key point information of two anatomical regions of interest according to the image data;
402: determining, according to the key point information, two direction indication lines representing the two anatomical regions of interest; and
403: calculating a second included angle between the two anatomical regions of interest according to the two direction indication lines.

In some embodiments, the OpenPose model is used as an example. At 401, the (matched) image data is inputted into the OpenPose model, a feature is extracted, and key point information is detected. The key point information is defined by the OpenPose model. The key point information may be represented by using coordinates (two-dimensional pixel coordinates) in an image coordinate system, or may be represented by using three-dimensional spatial coordinates in a camera coordinate system. For coordinate transformation, reference may be made to the related art, and details are not described herein again.

In some embodiments, at 402, linear fitting is performed on the key point information to obtain two fitted straight lines representing the two anatomical regions of interest. Coordinate transformation is performed on the two fitted straight lines according to parameters of the image capture apparatus to obtain two direction indication lines corresponding to the two fitted straight lines. To be specific, the key points are connected (by means of linear fitting) according to the setting of the model, particularly for an anatomical region such as a joint, to obtain two fitted straight lines representing the two anatomical regions of interest. As shown in FIG. 5(a), the key points are k1, k2, k3, and k4, the two fitted straight lines are L1 and L2. L1 may represent the ankle position, and L2 may represent the foot position.

In some embodiments, for the above key point information, the fitted straight lines are obtained in the image coordinate system or the camera coordinate system. To assist in positioning, coordinate transformation needs to be performed on the two fitted straight lines according to the parameters of the image capture apparatus, so that coordinates are transformed into the world coordinate system (Eulerian coordinate system), or into the spherical coordinate system, to obtain two direction indication lines (that is, spatial vector lines a1 and b1) corresponding to the two fitted straight lines. FIG. 5(b) is a schematic diagram of the two direction indication lines in the world coordinate system according to an embodiment of the present application. For a coordinate transformation method, reference may be made to the related art. The embodiments of the present application are not limited thereto.

For the direction indication line determined by using the method in FIG. 4, a cosine angle inverse function between the vector lines a1 and b1 is calculated to obtain the second included angle. For example, the included angle between the two direction indication lines is calculated by using formula (1), and the included angle γ is used as the second included angle (for example, the included angle between the foot and the ankle).

$$γ = arccos ( \frac{a 1 * b 1}{\vert a 1 \vert * \vert b 1 \vert} ) \qquad Formula ( 1 )$$

In some embodiments, to avoid false detection, optionally, the method may further include: calculating a slope of the two fitted straight lines, and calculating a normalized distance according to the slope. For a method for calculating the slope (or the gradient) and the normalized distance, reference may be made to the related art. The embodiments of the present application are not limited thereto.

In some embodiments, when the normalized distance is greater than or equal to a threshold, coordinate transformation is performed on the two fitted straight lines according to the parameters of the image capture apparatus to obtain the two direction indication lines corresponding to the two fitted straight lines. When the normalized distance is less than the threshold, it indicates that the two fitted straight lines may also be regarded as one straight line, and therefore, no included angle needs to be calculated.

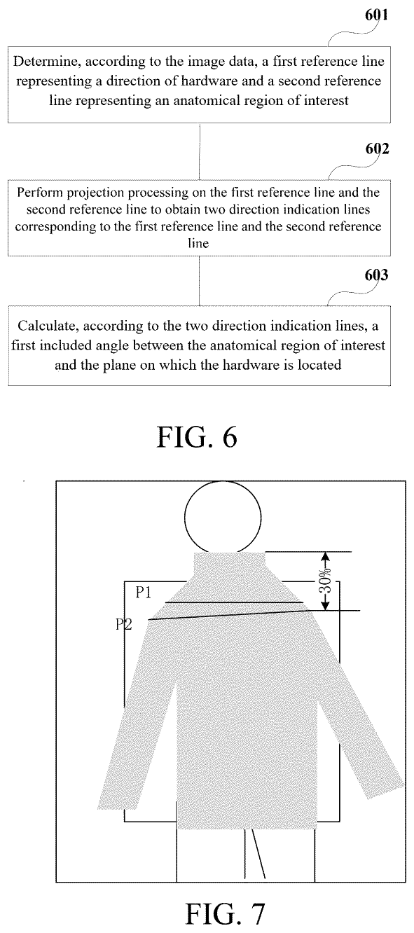

FIG. 6 is a schematic diagram of a method for calculating a first included angle according to an embodiment of the present application. As shown in FIG. 6, the method includes:
601: determining, according to the image data, a first reference line representing a direction of the hardware and a second reference line representing an anatomical region of interest;
602: performing projection processing on the first reference line and the second reference line to obtain two direction indication lines corresponding to the first reference line and the second reference line; and
603: calculating, according to the two direction indication lines, a first included angle between the anatomical region of interest and the plane on which the hardware is located.

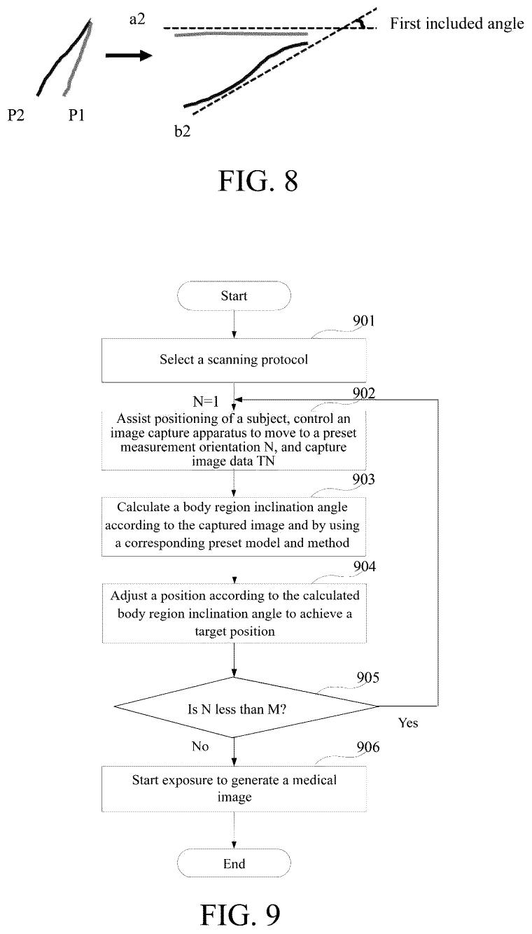

In some embodiments, the position of the first reference line, in the image data, that is parallel to an edge (for example, a horizontal edge) of the hardware of the medical imaging system or that is perpendicular to a plane on which the hardware is located may be determined through coordinate transformation according to the position of the image capture apparatus (the suspension apparatus), the coordinates of the hardware in the medical imaging system, and the parameters of the image capture apparatus. As shown in P1 in FIG. 8, P1 is parallel to a detector edge of the medical imaging system.

In some embodiments, the mask R-CNN model is used as an example. The (matched) image data is inputted into the mask R-CNN model, and an area matching a preset mask is determined. The second reference line is determined, in the area matching the preset mask, according to the height or width percentage of the anatomical region of interest. The preset mask and the percentage may be set as required. The embodiments of the present application are not limited thereto. The second reference line is determined by determining the height or the width formed by the maximum and minimum values of the upper, lower, left, and right edges in the area matching the preset mask, and further by using the height or width percentage of the anatomical region of interest. Considering the depth information, the second reference line is not necessarily a straight line.

FIG. 7 is a schematic diagram of an area matching a preset mask according to an embodiment of the present application. An area of the human body from the neck to the waist is set as the preset mask (a gray area in the figure). The mask R-CNN model may determine the area matching the preset mask in the image data. Even if the sizes and positions of the area corresponding to different subjects are different, the position of the scapula is located at the percentage of 30% in the height direction of the area matching the preset mask. A line of the percentage of 30% in the height direction of the area matching the preset mask is used as a second reference line P2. P2 may represent a position of the shoulder joint. An example in which the preset mask is the area from the neck to the waist, and the percentage is 30% is used above for description, but the embodiments of the present application are not limited thereto. For example, the preset mask is the thigh area, a line of the percentage of 50% in the width direction of the area matching the preset mask is used as a second reference line P2, and the second reference line P2 may also be regarded as a center line of the thigh area.

In some embodiments, at 602, projection processing is performed on the first reference line and the second reference line in a normal vector direction of an orientation of interest. The normal vector direction of the orientation of interest may be preset, but the embodiments of the present application are not limited thereto. For example, the normal vector of the orientation of interest may be a normal vector of the plane on which the first reference line and the second reference line are located. No further examples are provided herein. On the plane, projection processing is performed on the first reference line and the second reference line in the normal vector direction of the orientation of interest, to obtain two projection lines corresponding to the first reference line and the second reference line. As shown in FIG. 8, an example in which the normal vector of the orientation of interest is the normal vector of the plane on which the first reference line and the second reference line are located is used. The projection processing is equivalent to generating a top view of the first reference line and the second reference line, and two projection lines (two direction indication lines) are obtained through projection on the top view. Only an example is used herein for description. The first reference line and the second reference line may alternatively be projected onto another reference plane to calculate a 2D included angle. The embodiments of the present application are not limited thereto.

For the projection lines determined by using the method in FIG. 6, approximate lines a2 and b2 of the two projection lines are determined on the top view (as shown in FIG. 8), and a 2D included angle between the two approximate lines is calculated on the top view, and the included angle is used as the first included angle (for example, an included angle between the shoulder joint and the plane on which the detector is located).

The OpenPose model and the mask R-CNN model are used above as an example to describe how to determine the direction indication line and the body region inclination angle, but the embodiments of the present application are not limited thereto.

In some embodiments, optionally, the method may further include:
304: displaying, in the image data, at least one of the direction indication line, the body region inclination angle, a reference line, a reference angle, and a posture adjustment indication in a superimposed manner.

For example, the above displaying in a superimposed manner is performed for each frame of image data (for example, on an optical image) of a real-time video stream captured by the image capture apparatus.

For example, the direction indication line and the calculated body region inclination angle may be displayed in the image data in a superimposed manner to determine a current position of the anatomical region of interest. At least one of the reference line and the reference angle may be displayed in the image data in a superimposed manner. The reference line and the reference angle may represent a target position of the anatomical region of interest. When the posture of the subject changes, the direction indication line and the body region inclination angle are also changed and displayed in real time, and the operator may adjust the direction indication line so as to coincide with the reference line or adjust the body region inclination angle so as to match the size of the reference angle, thereby completing correct positioning. The direction indication line and the reference line may be displayed in different line types (for example, a dashed line and a solid line, respectively), and the reference angle and the body region inclination angle may also be displayed in different display manners. The embodiments of the present application are not limited thereto.

For example, a posture adjustment indication is displayed in the image data in a superimposed manner. The indication may be an arrow or the like for adjusting the direction, and the arrow may assist the user in performing correct positioning.

In some embodiments, the method may further include: displaying at least one of the direction indication line, the body region inclination angle, the reference line, the reference angle, and the posture adjustment indication on the body of the subject. For example, a projection apparatus may be added to the medical imaging apparatus (for example, to the beam limiter 117 or the tube assembly 115). The at least one of the direction indication line, the body region inclination angle, the reference line, the reference angle, and the posture adjustment indication is projected onto the body of the subject by using the projection apparatus, to assist the user in performing positioning in a more intuitive manner.

In some embodiments, optionally, the method may further include: issuing, by the medical imaging system, a notification when the positioning is correct. For example, the notification is issued by displaying a display window in a graphical user interface or by using a sound. The embodiments of the present application are not limited thereto.

FIG. 9 is a schematic diagram of a medical imaging method according to an embodiment of the present application. As shown in FIG. 9, the method includes:
901: selecting a scanning protocol; in this way, (M) angle measurement orientations and a type of an included angle that needs to be calculated, which are set corresponding to the scanning protocol, may be determined, and a corresponding relationship between the scanning protocol and each of the angle measurement orientation, the type of the included angle, and the selected model and calculation method is preset;
902: assisting the positioning of the subject, controlling the image capture apparatus to move to a preset angle measurement orientation N, and capturing image data TN; (initially, N=1)
903: calculating a body region inclination angle according to the captured image and by using a corresponding preset model and method;
904: adjusting a position according to the calculated body region inclination angle to achieve a target position;
905: when N is less than M, N=N+1, returning to 902, and when N is greater than or equal to M, performing 906; and
906: starting exposure to obtain a medical image.

For example, the scanning protocol is a patella axis—sunrise. For this protocol, two angle measurement orientations (the bottom view and the side view) that need to be set are preset. The first included angle is calculated by using the OpenPose model and the method corresponding to the OpenPose model on the bottom view, and the second included angle is calculated by using the mask R-CNN model and the method corresponding to the mask R-CNN model on the side view. First, the positioning of the subject is assisted, the image capture apparatus is controlled to move to angle measurement orientation 1 (the side view), image data T1 is captured, a second included angle is calculated according to the image data T1 and by using the mask R-CNN model and the method corresponding to the mask R-CNN model, and a position is adjusted according to the calculated second included angle, so as to achieve a target position between a thigh and a leg. The image capture apparatus is controlled to move to angle measurement orientation 2 (the bottom view), image data T2 is captured, a first included angle is calculated according to the image data T2 and by using the OpenPose model and the method corresponding to the OpenPose model, and a position is adjusted according to the calculated first included angle, so as to achieve a line of a patella apex and a tibiofibular bone perpendicular to the handheld detector plane. Then, exposure is started to obtain a medical image.

It should be noted that the foregoing accompanying drawings merely schematically illustrate embodiments of the present application, but the present application is not limited thereto. For example, the order of execution of operations may be appropriately adjusted. In addition, some other operations may be added or some operations may be omitted. Alternatively, operations may be added. Those skilled in the art can make appropriate variations according to the above content, rather than being limited by the disclosure of the foregoing accompanying drawings.

The above embodiments merely provide illustrative descriptions of the embodiments of the present application. However, the present application is not limited thereto, and appropriate variations may be made on the basis of the above embodiments. For example, each of the embodiments described above may be used independently, or one or more among the above embodiments may be combined.

In the embodiments of the present application, the body region inclination angle is calculated by using the image data captured by the image capture apparatus, so as to assist the operator in performing the positioning in a more quantitative and accurate manner, and reduce the number of exposures and the positioning time.

In addition, not only an included angle between anatomical regions of interest, but also an included angle between an anatomical region of interest and system hardware (a surface of the detector, the bedplate, or the wall stand) may be determined according to the image data, so that the application scenarios are more extensive.

In addition, the type and the number of models used are related to the scanning protocol, at least two different scanning protocols correspond to different types or numbers of models, and the corresponding models and the types of included angles to be calculated may be preset for the scanning protocol, so that the included angles may be determined by using a plurality of models, more suitable models may be selected for the different scanning protocols to determine the included angles, the implementation manner is more flexible and intelligent, and the workflow is more automated.

In addition, at least one of the direction indication line, the body region inclination angle, the reference line, the reference angle, and the posture adjustment indication may alternatively be displayed in the image data in a superimposed manner, thereby more intuitively assisting the user in performing the positioning.

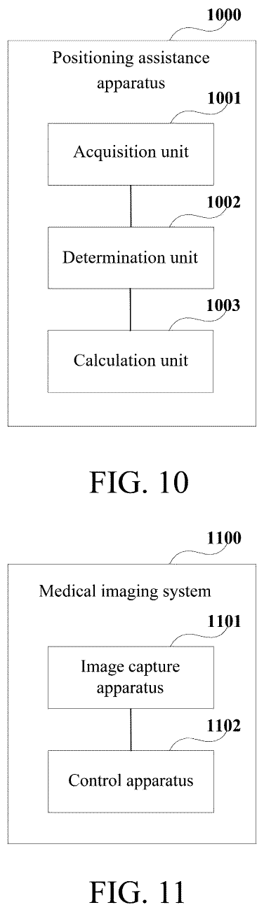

Further provided in an embodiment of the present application is a positioning assistance apparatus. FIG. 10 is a schematic diagram of a positioning assistance apparatus according to an embodiment of the present application. As shown in FIG. 10, the apparatus 1000 includes:

an acquisition unit 1001, configured to obtain image data that is captured via an image capture apparatus and that includes a subject, the image data including optical image data and depth image data;

a determination unit 1002, configured to determine, based on the image data, a direction indication line representing at least one anatomical region of interest; and

a calculation unit 1003, configured to calculate a body region inclination angle based on the direction indication line, the body region inclination angle including at least one of a first included angle and a second included angle, the first included angle representing an included angle between an anatomical region of interest and a plane on which hardware of a medical imaging system is located, and the second included angle representing an included angle between two anatomical regions of interest.

For the implementations of modules of the foregoing units, reference may be made to 301 to 303, and repeated parts are not described again.

Optionally, the apparatus may further include: a display unit (not shown in the figure), configured to display a graphical user interface, the graphical user interface including an area for displaying the image data; and further display, in the image data, at least one of the direction indication line, the body region inclination angle, a reference line, a reference angle, and a posture adjustment indication in a superimposed manner.

Further provided in an embodiment of the present application is a medical imaging system. FIG. 11 is a schematic diagram of a medical imaging system according to an embodiment of the present application. As shown in FIG. 11, the system 1100 includes:

an image capture apparatus 1101, configured to capture a subject to obtain image data including the subject, the image data including optical image data and depth image data; and

a control apparatus 1102, connected to the image capture apparatus and configured to perform the positioning assistance method in the foregoing embodiments.

For the embodiments of the image capture apparatus 1101 and the control apparatus 1102, reference may be made to the foregoing embodiments, which are not repeated herein. The medical imaging system includes, but is not limited to: a computed tomography (CT) system, a magnetic resonance imaging (MRI) system, a C-arm imaging system, a positron emission computed tomography (PET) system, a single photon emission computed tomography (SPECT) system, an ultrasound system, an X-ray imaging system, or any other suitable medical imaging system.

In some embodiments, the control apparatus 1102 may be configured separately from a controller of the medical imaging system. For example, the control apparatus 1102 is configured as a chip or the like connected to the controller of the medical imaging system, and the chip and the controller may control each other. Alternatively, functions of the control apparatus 1102 may be integrated into the controller of the medical imaging system. The embodiments of the present application are not limited thereto.

In some embodiments, the control apparatus 1102 includes a computer processor and a storage medium. The storage medium records a program for predetermined data processing to be executed by the computer processor. For example, the storage medium may store a program used to implement subject tracking for medical imaging. The storage medium may include, for example, a ROM, a floppy disk, a hard disk, an optical disc, a magneto-optical disc, a CD-ROM, or a non-volatile memory card.

The medical imaging system may further include other structural components not shown in the figure. For details, reference may be made to the related art. For example, a display apparatus (not shown in the figure) is configured to display a graphical user interface, the graphical user interface including an area for displaying the image data; and the display apparatus further displaying, in the image data, at least one of the direction indication line, the body region inclination angle, a reference line, a reference angle, and a posture adjustment indication in a superimposing manner. In addition, other structural components may be further included. The embodiments of the present application are not limited thereto. For example, reference may be made to FIG. 1, and details are not described herein again.

Further provided in an embodiment of the present application is a computer-readable program, wherein the program, when executed in an apparatus or a medical imaging system, causes a computer to execute, in the apparatus or the medical imaging system, the positioning assistance method according to the foregoing embodiments.

Further provided in an embodiment of the present application is a computer program product, including at least a computer-readable program, wherein the computer-readable program causes a computer to execute, in an apparatus or a medical imaging system, the positioning assistance method according to the foregoing embodiments.

The above apparatus and method of the present application may be implemented by hardware, or may be implemented by hardware in combination with software. The present application relates to such a computer-readable program that when executed by a logic component, the program causes the logic component to implement the foregoing apparatus or a constituent component, or causes the logic component to implement various methods or steps described above. The present application further relates to a storage medium for storing the above program, such as a hard disk, a magnetic disk, an optical disc, a DVD, a flash memory, etc.

The method/apparatus described with reference to the embodiments of the present application may be directly embodied as hardware, a software module executed by a processor, or a combination of the two. For example, one or more of the functional block diagrams and/or one or more combinations of the functional block diagrams shown in the figures may correspond to either software modules or hardware modules of a computer program flow. The foregoing software modules may respectively correspond to the steps shown in the figures. The foregoing hardware modules may be implemented, for example, by consolidating the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, or may be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for implementing the functions described in the present application. The one or more functional blocks and/or the one or more combinations of the functional blocks shown in the drawings may alternatively be implemented as a combination of computing devices, such as a combination of a DSP and a microprocessor, a plurality of microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and these variations and modifications also fall within the scope of the present application.

1. A positioning assistance method, the method comprising:
obtaining image data captured via an image capture apparatus and comprising a subject, the image data comprising optical image data and depth image data;
determining, according to the image data, a direction indication line representing at least one anatomical region of interest; and
calculating a body region inclination angle according to the direction indication line, the body region inclination angle comprising at least one of a first included angle and a second included angle, the first included angle representing an included angle between an anatomical region of interest and a plane on which hardware of a medical imaging system is located, and the second included angle representing an included angle between two anatomical regions of interest.

2. The method according to claim 1, wherein the steps of determining, according to the image data, a direction indication line representing at least one anatomical region of interest, and calculating a body region inclination angle according to the direction indication line comprise:
determining, according to the image data, a first reference line representing a direction of the hardware and a second reference line representing an anatomical region of interest;
performing projection processing on the first reference line and the second reference line to obtain two direction indication lines corresponding to the first reference line and the second reference line; and
calculating, according to the two direction indication lines, the first included angle between the anatomical region of interest and the plane on which the hardware is located.

3. The method according to claim 2, wherein the first reference line is a straight line in the image data parallel to an edge of the hardware, or perpendicular to the plane on which the hardware is located.

4. The method according to claim 2, wherein the step of performing projection processing on the first reference line and the second reference line comprises: performing projection processing on the first reference line and the second reference line in a normal vector direction of an orientation of interest.

5. The method according to claim 2, wherein the determining, according to the image data, a second reference line representing an anatomical region of interest comprises:
determining, according to the image data, an area matching a preset mask; and
determining, in the area matching the preset mask, the second reference line according to the height or width percentage of the anatomical region of interest.

6. The method according to claim 2, wherein the step of calculating, according to the two direction indication lines, the first included angle comprises:
determining approximate lines for the two direction indication lines; and
calculating an included angle between the two approximate lines, and using the included angle as the first included angle.

7. The method according to claim 1, wherein the steps of determining, according to the image data, a direction indication line representing at least one anatomical region of interest, and calculating a body region inclination angle according to the direction indication line comprise:
determining key point information of two anatomical regions of interest according to the image data;
determining, according to the key point information, two direction indication lines representing the two anatomical regions of interest; and
calculating the second included angle between the two anatomical regions of interest according to the two direction indication lines.

8. The method according to claim 7, wherein the step of determining, according to the key point information, two direction indication lines representing the two anatomical regions of interest comprises:
performing linear fitting on the key point information to obtain two fitted straight lines representing the two anatomical regions of interest; and
performing coordinate transformation on the two fitted straight lines according to a parameters of the image capture apparatus to obtain two direction indication lines corresponding to the two fitted straight lines.

9. The method according to claim 8, further comprising:
calculating a slope of the two fitted straight lines;
calculating a normalized distance according to the slope;
and when the normalized distance is greater than or equal to a threshold, performing coordinate transformation on the two fitted straight lines according to the parameters of the image capture apparatus to obtain the two direction indication lines corresponding to the two fitted straight lines.

10. The method according to claim 1, further comprising:
performing matching processing on the optical image data and the depth image data;
and determining, according to the matched image data, the direction indication line representing the at least one anatomical region of interest.

11. The method according to claim 1, further comprising:
determining, according to a scanning protocol, a type of the body region inclination angle that needs to be calculated and the number of angle measurement orientations.

12. The method according to claim 1, further comprising:
displaying, in the image data, at least one of the direction indication line, the body region inclination angle, a reference line, a reference angle, and a posture adjustment indication in a superimposed manner.

13. A medical imaging system, comprising:
an image capture apparatus, capturing a subject to obtain image data comprising the subject, the image data comprising optical image data and depth image data; and
a control apparatus, connected to the image capture apparatus and configured to:
obtain the image data from the image capture apparatus;
determine, according to the image data, a direction indication line representing at least one anatomical region of interest; and
calculate a body region inclination angle according to the direction indication line, the body region inclination angle comprising at least one of a first included angle and a second included angle, the first included angle representing an included angle between an anatomical region of interest and a plane on which hardware of a medical imaging system is located, and the second included angle representing an included angle between two anatomical regions of interest.

14. The system according to claim 13, further comprising:
a display apparatus, displaying a graphical user interface, the graphical user interface comprising an area for displaying the image data,
and the display apparatus further displaying, in the image data, at least one of the direction indication line, the body region inclination angle, a reference line, a reference angle, and a posture adjustment indication in a superimposed manner.
